(19) United States
(12) Patent Application Publication
(10) Pub. No.: US 2005/0131238 A1
(43) Pub. Date: Jun. 16, 2005

(54) Spermicides

(75) Inventors: Suren Solanki, Cambridge (GB); William Duncan Potter, Cambridge (GB); Lorraine Anderson, Edinburgh (GB)

Correspondence Address: STERNE, KESSLER, GOLDSTEIN & FOX PLLC, 1100 NEW YORK AVENUE, N.W., WASHINGTON, DC, 20005, US

(73) Assignee: SSL International plc, Cambridgeshire CB4 0GZ (GB)

(21) Appl. No.: 10/493,471

(22) PCT Filed: Oct. 24, 2002
(86) PCT No.: PCT/GB02/04813

(30) Foreign Application Priority Data
Oct. 25, 2001 (GB) 0125659.3

(51) Int. Cl. C07D 27/448

(52) U.S. Cl. 548/548; 514/718

(57) ABSTRACT

The present invention relates to the use of maleimide derivatives as spermicidal agents. In particular it relates to the use of a maleimide compound of formula (I): wherein R is any group with the proviso that the maleimide is not N-ethylmaleimide, as a spermicide. The invention further provides novel these compounds and their use in medicine. The invention also relates to spermicidal formulations containing maleimide derivatives.

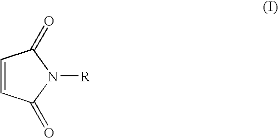

The present invention relates to the use of maleimide derivatives as spermicidal agents. In particular it relates to the use of maleimide derivatives as contact spermicides. The invention further provides novel compounds and their use in medicine. The invention also relates to spermicidal formulations containing maleimide derivatives.

Spermicides are chemical products which inactivate or kill sperm. In use, they are generally applied topically to the vagina prior to sexual intercourse. Spermicides are considered to be a convenient form of contraception as they are widely available without prescription and they can be self-administered. In contrast to the contraceptive pill, spermicides do not need to be applied on a regular basis, but only prior to intercourse, their effect is temporary and they have no hormonal side effects. Spermicides may be used alone, but are often used in combination with barrier methods of contraception.

Currently available spermicides include nonoxynol-9, octoxynol-9, menfegol and benzalkonium chloride, which are all classified as detergent spermicides. It has been suggested that the detergent-type spermicides can cause lesions of the vaginal and cervical epithelia.

Maleimides are a well known class of chemical compounds. Maleimide derivatives are known to have a wide variety of utilities, and find application in the biological and medicinal fields, as well as in general industrial use, for example in the preparation of polymers and inks.

Thus for example, U.S. Pat. No. 3,129,225 (United States Vitamin and Pharmaceutical Corporation) describes maleimido betaines said to have antimicrobial and antiparasitic activity as well as pharmacological activity, in particular the reduction of serum cholesterol.

German ALS 1,140,192 (United States Rubber Company) describes a novel bismaleimide with a CH₂—O—CH₂ bridge, and its use in vulcanisation, cross-linking of polymers and as fungicide for tomatoes.

U.S. Pat. No. 4,876,358 (Texaco Inc.) describes novel bismaleimides having a (poly)oxyethylene bridge, for use in preparing polymers.

U.S. Pat. No. 5,552,396 and WO 97/19080 (Eli Lilly) describe classes of N,N-bridged bisindolylmaleimides which are said to be inhibitors of protein kinase C, and to be useful for the treatment of diabetes, ischemia, inflammation, cns disorders, cardiovascular disease, dermatological disease and cancer.

U.S. Pat. No. 5,380,764 (Goedecke A G) describes bis-indolemaleinimides,said to be protein kinase C inhibitors useful for treating diseases of heart or blood vessels, inflammatory processes, allergies, cancer and degenerative damage of cns, diseases of the immune system and viral diseases, including diseases caused by retroviruses HTLV-I, -II, -III.

WO 96/09406 (The Government of the United States of America, represented by the Secretary of the Department of Health and Human Services) describes the use of disulfides, maleimides and a variety of other electron acceptor compounds in inactivating HIV-1 and other retroviruses.

Igarashi et al., J. Industrial Microbiology, 6 (1990) pp223-225, report that various N-alkylmaleimides show antibacterial and antifungal activity.

Reyes et al, Archives of Andrology, 7, (1990), pp159-168, discloses the use of N-(4-carboxy-3-hydroxy-phenyl)-maleimide (CPhM) to reduce the fertilising capacity of sperm. Incubation with a 10 mM CPhM solution for 10 minutes resulted in the fertilising capacity of sperm being decreased from 80% to 17%. 10 minutes exposure is outside what is considered to be a clinically relevant timescale for a spermicide, that is, between 20 and 40 seconds following the addition of a putative spermicide (Sander F V and Cramer: A practical method for testing the spermicidal action of chemical contraceptives, Hum. Fertil., 1941, 6, 134-153 and Kleinman R L in “IPPF Medical Handbook” 2^{nd} edition, London, 1964, International Planned Parenthood Federation).

There is therefore a continuing need for spermicidal compounds which act quickly and efficiently to inactivate or kill sperm.

We have now surprisingly found that the maleimide derivatives of the present invention reduce human sperm motility. The compounds are therefore useful as spermicides. Furthermore, results from cell cytotoxicity assays suggest that the compounds do not cause significant damage to the vaginal and cervical epithelium.

In a first aspect therefore the present invention provides the use of a maleimide compound of the general formula (I) as a spermicide 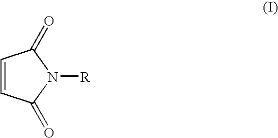
wherein R is any group with the proviso that the maleimide is not N-ethylmaleimide. The term maleimide compound includes all derivatives, salts and substituted maleimides, including optionally substituted mono-maleimides, bis-maleimides and poly-maleimides.

The maleimide compound is preferably represented by the general formula (Ia) 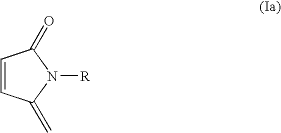
wherein
R represents a group A, which itself is represented by (R¹)ₙ—(R²)ₚ—(R³)_{q}, wherein each of R¹, R² and R³ is independently selected from:
an acyclic aliphatic group having one or from 3-20 carbon atoms;
a polyalkylene glycol group having an average molecular weight in the range 100 to 1000;
a cyclic aliphatic group having from 4-20 carbon atoms;
a monocyclic or polycyclic aromatic hydrocarbon group having from 6-18 ring carbon atoms;
a non-aromatic heterocyclic group having from 3 to 8 ring atoms, or
a monocyclic or polycyclic aromatic heterocyclic group having from 5 to 20 ring atoms;
and n, p and q each represent 0 or 1 such that the sum of n+p+q=1-3;
or R represents the group A substituted by one or more groups independently selected from: 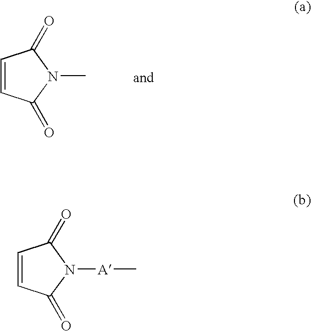
   wherein A′, which may be the same as or different from A, represents a group (R¹)ₙ—(R²)ₚ—(R³)_{q}, wherein R¹, R², R³ , n, p and q are as defined above;
or a salt, ester or other physiologically equivalent derivative thereof.

In the compounds of formula (I) above an acyclic aliphatic group or moiety may be branched or unbranched alkyl, which alkyl group may be optionally interrupted by one or more oxygen atoms; C₂₋₂₀ alkenyl; or C₂₋₂₀ alkynyl; any of which may be optionally substituted by one or more substituents selected from the group consisting of halogen, cyano, nitro, —C(O)R⁴, —CO₂R⁴, —OR⁴, —SR⁴—SOR⁴, —SO₂R⁴, —NR⁴R⁵, —C(O)NR⁴R⁵; wherein R⁴ and R⁵ independently represent hydrogen, C₁₋₈alkyl, aryl or arylC₁₋₈alkyl.

For the purposes of the present invention, alkyl groups may have from 1 or 3 to 20 carbon atoms, preferably from 1 or 3 to 15 carbon atoms, more preferably from 1 or 3 to 10 carbon atoms. Alkenyl groups may have from 2 to 20 carbon atoms, preferably from 2 to 15 carbons atoms, more preferably from 2 to 10 carbon atoms. Alkynyl groups may have from 2 to 20 carbon atoms, preferably from 2 to 15 carbon atoms, more preferably from 2 to 10 atoms.

A polyalkyleneglycol group may be a polyethylene glycol (PEG) or a polypropylene glycol which may be represented by the formulae —(OCH₂CH₂)ₘOH or —(OCH₂CH₂CH₂)ₘOH, wherein m is in the range 4 to 20. It will be appreciated that polyalkylene glycols are generally a mixture of chain lengths and are therefore conventionally described in terms of average molecular weight and average values of m. Thus for example PEG 200 has molecular weight in the range 190-210 and an average value of m=4 (The Merck Index, Eleventh edition).

Aryl is phenyl optionally substituted by one or more substituents selected from the group consisting of halogen, cyano, nitro, —C(O)R⁶, —CO₂R⁶, —OR⁶, —SR⁶—SOR⁶, —SO₂R⁶, —NR⁶R⁷, —C(O)NR⁶R⁷ wherein R⁶ and R⁷ independently represent hydrogen, or C₁₋₈alkyl.

A cyclic aliphatic group or moiety may be cycloalkyl or cycloalkenyl, either of which may be optionally substituted by one or more substituents selected from the group consisting of halogen, cyano, nitro, —C(O)R⁴, —CO₂R⁴, —OR⁴, —SR⁴—SOR⁴, —SO₂R⁴, —NR⁴R⁵, —C(O)NR⁴R⁵; wherein R⁴ and R⁵ are as defined above.

Cycloalkyl groups of the present invention may have from 3 to 20 carbon atoms, preferably from 3 to 10 carbon atoms, more preferably from 3 to 6 carbon atoms. Cycloalkenyl groups of the present invention may have from 3 to 20 carbon atoms, preferably from 3 to 10 carbon atoms, more preferably from 3 to 6 carbon atoms.

Aromatic hydrocarbon groups and moieties include for example optionally substituted unsaturated monocyclic, fused bicyclic or tricyclic rings of up to 18 carbon atoms, such as phenyl and naphthyl, and partially saturated bicyclic or tricyclic rings such as tetrahydro-naphthyl. Aromatic groups also include linked aromatic rings, such as a biphenyl group. The aromatic rings may also be linked by heteroatoms, e.g. O, S or NR⁴. Examples of substituents which may be present on an aromatic group or moiety include one or more of halogen, alkyl, haloalkyl, cyano, nitro, —C(O)R⁴, —CO₂R⁴, —OR⁴, —SR⁴—SOR⁴, —SO₂R⁴, —NR⁴R⁵, or —C(O)NR⁴R⁵. Substituents on the aromatic ring are preferably in the ortho or para position with respect to the maleimido group.

An aromatic heterocyclic group or moiety may be unsaturated or partially saturated, for example an optionally substituted 5- or 6-membered heterocyclic aromatic ring which may contain from 1 to 4 heteroatoms selected from O, N and S. The heterocyclic ring may optionally be fused to one or more phenyl rings. Examples of heteroaryl groups thus include furyl, thienyl, pyrrolyl, oxazolyl, oxazinyl, thiazolyl, imidazolyl, oxadiazolyl, thiadiazolyl, pyridyl, triazolyl, triazinyl, pyridazyl, pyrimidinyl, pyrazolyl, indolyl, indazolyl, benzofuranyl, benzothienyl, benzimidazolyl, benzoxazolyl, benzoxazinyl, quinoxalinyl, quinolinyl, quinazolinyl, cinnolinyl, benzothiazolyl, pyridopyrrolyl. Examples of substituents which may be present on a heterocyclic aromatic group include one or more of halogen, oxo, nitro, cyano, alkyl, haloalkyl, —C(O)R⁴, —CO₂R⁴, —OR⁴, —SR⁴—OR⁴, —SO₂R⁴, —NR⁴R⁵, or —C(O)NR⁴R⁵, wherein R⁴ and R⁵ are as defined above. Partially saturated derivatives of the aforementioned heteroaryl groups include 3-pyrroline and 1,2-dihydroquinoline.

Non-aromatic heterocyclic groups include pyrrolidinyl, tetrahydrofuryl, tetrahydrothienyl and piperidinyl.

Compounds of formula (I) for use according to the present invention thus include mono-maleimide derivatives of formula (II): 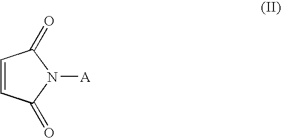
wherein A is as defined above,
and salts, esters and other physiologically equivalent derivatives thereof.

In the compounds of formula (II) R¹ preferably represents an aromatic hydrocarbon group, e.g. phenyl or naphthyl; or an aromatic heterocylic group e.g. thienyl or acridine. Optional substituents present on the phenyl group are preferably in the ortho or para position (relative to maleimido) and may be for example selected from amino, halogen, eg chlorine, C₁₋₄alkyl, nitro, phenoxy and phenylamino.

Compounds of formula (I) for use according to the invention also include those having two or more maleimide moieties, as represented by formula (III): 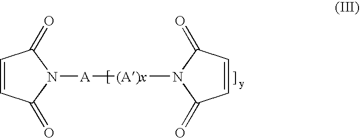
wherein A and A′, which may be the same or different, are as defined above,
x is 0 or 1 and y is 1-6.

In compounds of formula (III) when x is 1, it is preferred that the total number of atoms in the moieties A and A¹ does not exceed 50.

A preferred class of compounds for use according to the invention is that of bismaleimide derivatives represented by formula (IV): 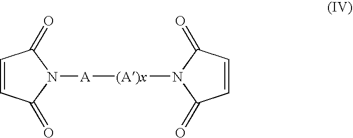
wherein A, A¹ and x are as defined above.

In the compounds of formula (IV) x preferably represents 0 and A is as defined above.

In the group A, R¹, R², R³, n, p and q are preferably selected such that A represents a C₁₋₈alkyl group, optionally interrupted by an oxygen atom;
a polyalkylene glycol group having an average molecular weight in the range 100 to 1000; or
a phenyl group; or
n and q are preferably each 1, p represents 0 or 1,R¹ and R³ preferably each represent phenyl and, when p is 1, R² preferably represents a C₁₋₈alkyl group, optionally interrupted by an oxygen atom; or
a polyalkylene glycol group having an average molecular weight in the range 100 to 1000.

When A represents phenyl the maleimido moieties are preferably in the para position relative to each other. When A represents biphenyl the bonds between the maleimido and phenyl groups and the bond between the two phenyl groups are also preferably in the para positions.

Maleimide derivatives for use according to the present invention are commercially available or can be prepared according to methods well known in the art.

A maleimide derivative for use according to the present invention may be administered as the sole active ingredient or may be administered in combination with one or more other maleimide derivatives as defined herein and/or in combination with one or more other therapeutic agents. In this context ‘in combination’ means that the compounds may be administered simultaneously or sequentially, and there may be an interval of time between administration of the two or more compounds. Therapeutic agents which may be administered in combination with one or more maleimide derivatives include antibacterial, antifungal, and antiviral agents.

For use as spermicides the maleimide derivatives may be formulated for administration to the reproductive tract using conventional methods known in the art. Thus for example they may be formulated as creams, gels including thermoreversible gels lubricants, pessaries, foaming tablets, aerosol foams, sprays, douches or films. The compounds may be incorporated into sustained release formulations, e.g. by micro-encapsulation, formulation with a bio-adhesive material, such as poly-carbophil, or other conventional means. Gels, creams and pessaries may, according to circumstance, be administered via an appropriate applicator. The formulations, in particular gels, creams and pessaries may be packaged with an appropriate applicator to facilitate administration.

In a second aspect therefore the present invention provides spermicidal preparations comprising a maleimide of formula (I).

Spermicidal preparations according to the present invention may also be incorporated into contraceptive devices. Thus for example they may be incorporated into vaginal, cervical or uterine devices such as vaginal sponges or rings, and diaphragms or used to lubricate and/or coat condoms. Spermicidal preparations used in this manner may be formulated for immediate and/or sustained release of the active compound(s) so as to provide controlled release over a period of time.

For use as a spermicide according to the present invention, a maleimide derivative may be administered at a dosage in the range 10-1000 mg, e.g. 30 to 1000 mg.

The present invention also provides contraceptive devices incorporating a spermicidal preparation comprising a maleimide of formula (I).

The third aspect of the invention provides a method of preventing conception comprising administering an effective amount of a maleimide compound as defined in the first aspect. The method is preferably for preventing conception in a mammal, more preferably in a human. The maleimide compound can be provided as a spermicidal composition or device as defined in the second aspect.

The fourth aspect of the invention provides the use of a maleimide compound as defined in the first aspect in the manufacture of an agent for the prevention of conception. The agent is a spermicide.

A fifth aspect of the invention provides a compound of formula (V) 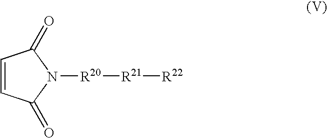
wherein R²⁰ is aryl, preferably phenyl;
R²¹ is —(OCH₂CH₂)ₙ— wherein n represents 3 to 15, preferably n is one of 4, 5, 6, 7, 8, 9, 10, 11, 12 and 13;
R²² is a group O—R²³ wherein R²³ is aryl or C₁₋₁₀ alkyl, preferably phenyl or C₁₋₄ alkyl, more preferably phenyl or methyl, wherein the aryl or alkyl group is optionally substituted with

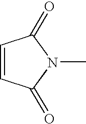

Preferred compounds of the fifth aspect are set out in the table below.

| Compound number | Structure |
|---|---|
| 13 | 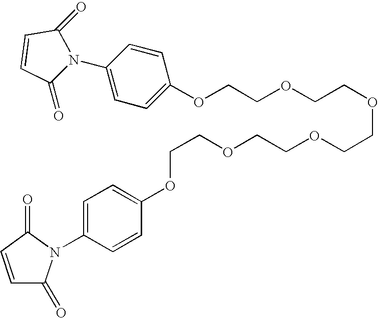 |
| 14 | 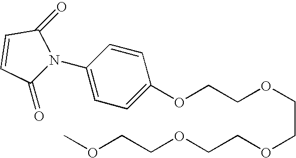 |
| 15 | 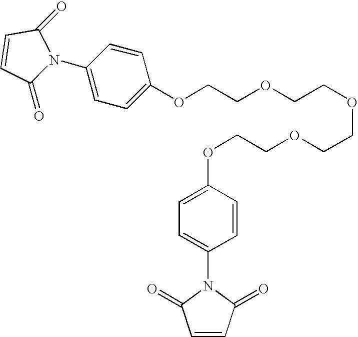 |
| 16 | 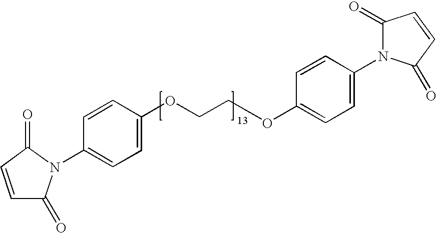 |

The fifth aspect of the invention also relates to salts, esters, amides or prodrugs of the compounds of formula V.

The compounds of the fifth aspect can be synthesised according to methods well known in the art. In particular, PEG of the appropriate chain length is initially converted to the aminoacyl derivative using conventional chemical synthesis. One example of such a chemical synthesis includes the coupling of PEG with p-fluoronitrobenzene in the presence of sodium hydride and DMF to produce a nitro-substituted aryl-PEG moiety. The nitro-subsituted aryl-PEG moiety is then reduced with a reducing agent such as palladium/carbon to form the required amine compound. The amine compound can then be converted into the maleimide compound of formula V by a number of methods known in the art. One example of such a method includes reacting the amine with maleic anhydride.

The sixth aspect of the invention provides a compound of formula V as defined in the fifth aspect of the invention for use in medicine. The compound of formula V is preferably provided as a spermicide.

All preferred features of different aspects of the invention apply to each other mutatis mutanalis.

The invention will be further illustrated by the following examples:

### EXAMPLES

Biological Testing

Methods

1. Evaluation of Human Sperm Motility (Spermicidal Efficacy)

The number of motile sperm (%) is rapidly determined (within 30s) following exposure to varying concentrations of spermicide using manual microscopy. Using non-linear regression analysis motility data from these screens is used to generate concentration response curves and subsequently an ED₅₀ value using GraphPad Prism software (San Diego, Calif., USA). The ED₅₀ value is the concentration of spermicide which produces 50% inhibition of sperm motility compared to control.

2. Determination of Cell Cytotoxicity (MTT Cell Viability Assay)

The effect of compounds on vaginal cell viability is assessed using the MTT assay. MTT is a tetrazolium salt which is converted by viable cells to an insoluble tetrazoliym salt. This colour conversion is measured by spectrophotometry. Data is then subjected to non-linear regression analysis to generate concentration response curves and subsequently an EC₅₀ value curves using GraphPad Prism software (San Diego, Calif., USA). The EC₅₀ value is the concentration of spermicide which produces 50% inhibition of cell viability compared to control.

3. Determination of Therapeutic Index

The therapeutic index is the ratio of cell cytoxicity EC₅₀ datum to the spermicidal efficacy ED₅₀ datum and is calculated using the data obtained in methods 1 and 2 above using the following formula:

$$Therapeutic Index = \frac{Cell Cytotoxicity [ {EC}_{50} ]}{Spermicidal efficacy [ {ED}_{50} ]}$$

Test Compounds

The following compounds of the invention were tested as set out in the above methods.

| Compound number | Structure |
|---|---|
| 1 | 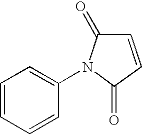 |
| 2 | 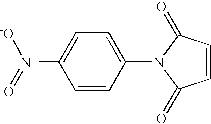 |
| 3 | 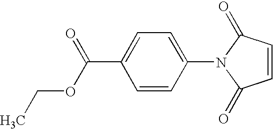 |
| 4 | 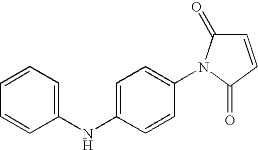 |
| 5 | 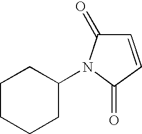 |
| 6 | 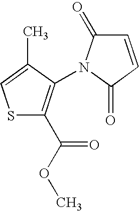 |
| 7 | 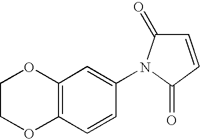 |
| 8 | 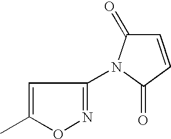 |
| 9 | 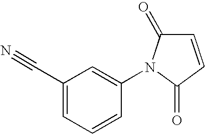 |
| 10 | 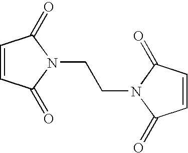 |
| 11 | 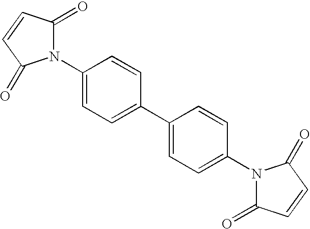 |
| 12 | 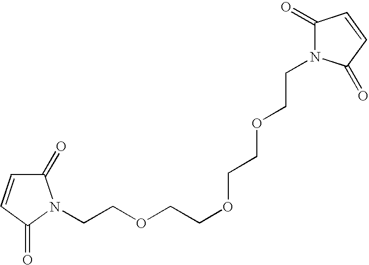 |
| 13 | 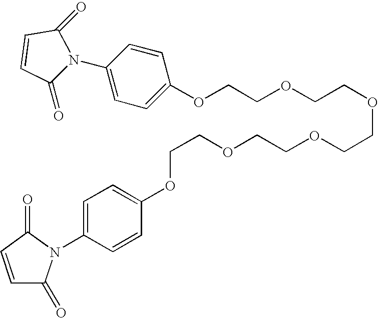 |
| 14 | 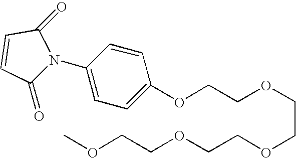 |
| 15 | 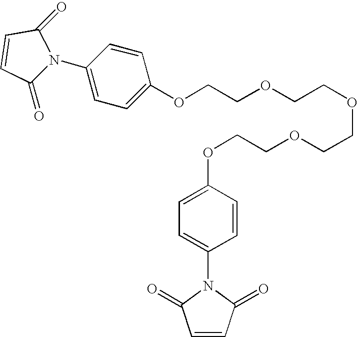 |
| 16 | 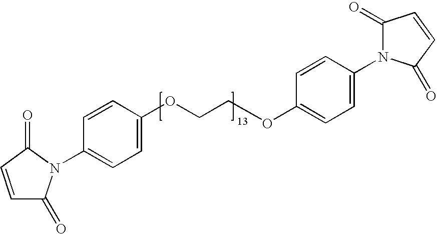 |

Compound 1 and 5 were obtained from Sigma-Aldrich Corp, compounds 2 and 10 from Tokyo Chemical Industry UK, compounds 3, 6, 7, 8 and 9 from Maybridge plc, compound 4 from Wako Pure Chemicals Industries, Ltd, compound 11 from Sigma-Aldrich library of a rare chemicals and compound 12 from Molecular Biosciences.

Results

1. Effect of Compounds Human Sperm Motility

TABLE 1
Effect of Maleimides on Human Sperm Motility. Sperm motility was
determined using manual microscopy. The concentration of each
compound required to inhibit sperm motility (ED₅₀) by 50% was
calculated from log concentration response curves using non-linear
regression analysis. The number of experiments is quoted ± SEM.

| | Inhibition Human |
|---|---|
| | Sperm Motility |
| Compound | (ED₅₀-μM) |
| 1 | 32 (±5) |
| | n = 3 |
| 2 | 26 (±7) |
| | n = 3 |
| 3 | 77 (±10) |
| | n = 3 |
| 4 | 31 (±3.5) |
| | n = 8 |
| 5 | 294 (±24) |
| | n = 6 |
| 6 | 26 (±1) |
| | n = 3 |
| 7 | 43 (±3) |
| | n = 3 |
| 8 | 79 (±16) |
| | n = 3 |
| 9 | 56 (±7) |
| | n = 3 |
| 10 | 4 (±0.5) |
| | n = 3 |
| 11 | 1.4 (±0.5) |
| | n = 4 |
| 12 | 10.5 (±3.5) |
| | n = 3 |
| 13 | 4.6 (±1.03) |
| | n = 4 |
| 14 | 149 (±19) |
| | n = 4 |
| 15 | 5 |
| | n = 2 |
| 16 | 34.4 (±7.2) |
| | n = 3 |

2. Effect of Compounds on Cell Cytotoxicity

TABLE 2
Effect of Maleimides on ME180 Cell Viability. ME180 cell viability
was determined using the MTT assay. The concentration of each
compound required to inhibit ME180 cell viability (EC₅₀) by 50%
was calculated from log concentration response curves using non-
linear regression analysis. The number of experiments is quoted ± SEM.

| | Inhibition ME 180 |
|---|---|
| | Cell Viability |
| Compound | (EC₅₀-μM) |
| 1 | 370 (±60) |
| | n = 3 |
| 2 | 600 (±101) |
| | n = 3 |
| 3 | 363 (±119.5) |
| | n = 3 |
| 4 | 268 (±126) |
| | n = 7 |
| 5 | 114 (±41) |
| | n = 3 |
| 6 | 88 (±28) |
| | n = 4 |
| 7 | 410 (±85) |
| | n = 4 |
| 8 | 858 (±142) |
| | n = 3 |
| 9 | 143 (±25) |
| | n = 3 |
| 10 | 516 (±106) |
| | n = 3 |
| 11 | >30 (±) |
| | n = 3 |
| 12 | 333 (±146) |
| | n = 4 |
| 13 | 125 (±23) |
| | n = 3 |
| 14 | 285 (±75) |
| | n = 3 |
| 15 | 153 n = 2 |
| 16 | 1890 (±1220) |
| | n = 5 |

3. Therapeutic Index

TABLE 3

| Compound | TI |
|---|---|
| 1 | 12 |
| 2 | 23 |
| 3 | 5 |
| 4 | — |
| 5 | 0.4 |
| 6 | 3 |
| 7 | 10 |
| 8 | 11 |
| 9 | 2.6 |
| 10 | 129 |
| 11 | >21 |
| 12 | 32 |
| 13 | 27 |
| 14 | 2 |
| 15 | 31 |
| 16 | 55 |

Conventional spermicides currently in use typically have therapeutic indices of 1 or less.

The foregoing description details specific compounds, compositions, methods and uses which can be employed to practice the present invention. However, those skilled in the art will know how to use alternative reliable methods for aiming at alternative embodiments of the invention which are herein encompassed.

1-14. (canceled)

15. A method for inactivating or killing sperm, or preventing conception, comprising administering to a mammal in need thereof a maleimide compound of formula (I) 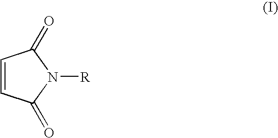
wherein R is any group, or a salt, ester or other physiologically equivalent derivative thereof, with the proviso that the maleimide is not N-ethylmaleimide.

16. The method of claim 15, wherein the maleimide compound is a compound of general formula (Ia): 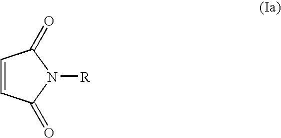
wherein:
R represents a group A, which itself is represented by (R¹)ₙ—(R²)ₚ—(R³)_{q}, wherein each of R¹, R² and R³ is independently selected from:
an acyclic aliphatic group having 1 or from 3-20 carbon atoms;
a polyalkylene glycol group having an average molecular weight in the range 100 to 1000;
a cyclic aliphatic group having from 4-20 carbon atoms;
a monocyclic or polycyclic aromatic hydrocarbon group having from 6-18 ring carbon atoms;
a non-aromatic heterocyclic group having from 3 to 8 ring atoms, or
a monocyclic or polycyclic aromatic heterocyclic group having from 5 to 20 ring atoms;
and n, p and q each represent 0 or 1 such that the sum of n+p+q=1-3;
or R represents the group A substituted by one or more groups independently selected from: 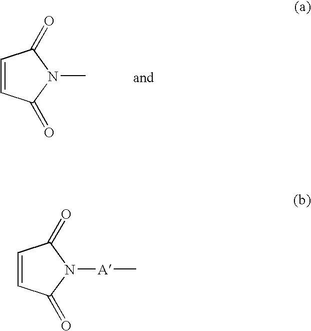
wherein A′, which may be the same as or different from A, represents a group (R¹)ₙ—(R²)ₚ—(R³)_{q}, wherein R¹, R², R³, n, p and q are as defined above;
or a salt, ester or other physiologically equivalent derivative thereof.

17. The method of claim 16, wherein the maleimide compound is a compound of formula (II): 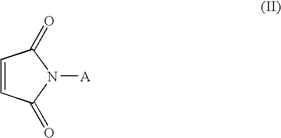
wherein A is as defined in claim 16, or a salt, ester or other physiologically equivalent derivative thereof.

18. The method of claim 16, wherein the maleimide compound is a compound of formula (III): 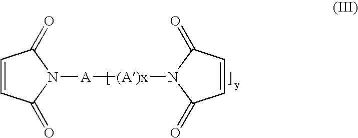
wherein A and A¹, which may be the same or different, are as defined in claim 16, x is 0 or 1 and y is 1-6, or a salt, ester or other physiologically equivalent derivative thereof.

19. The method of claim 16, wherein the maleimide compound is a compound of formula (IV): 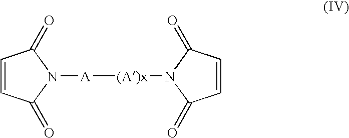
wherein A, A¹ and x are as defined in claim 16, or a salt, ester or other physiologically equivalent derivative thereof.

20. The method of claim 16, wherein the maleimide compound is compound of formula V: 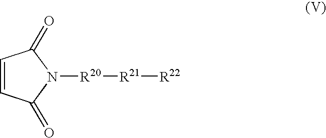
wherein R²⁰ is aryl;
R²¹ is —(OCH₂CH₂)ₙ— wherein n is 15;
R²² is a group O—R²³ wherein R²³ is aryl; or
R²¹ is —(OCH₂CH₂)ₙ wherein n is 3 to 15;
R²² is a group O—R²³ wherein R²³ is C₁₋₁₀ alkyl,
wherein the aryl or alkyl group is optionally substituted with 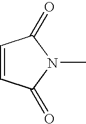
or a salt, ester or other physiologically equivalent derivative thereof.

21. The method of claim 16, wherein the maleimide compound is selected from any one of: 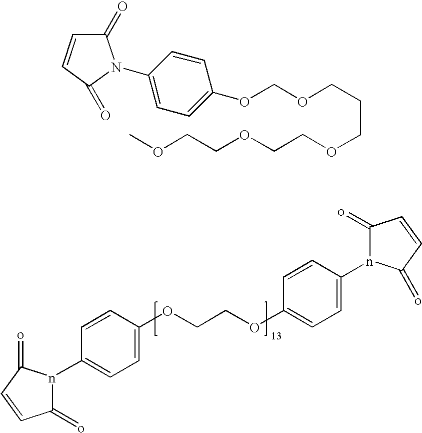
or a salt, ester or other physiologically equivalent derivative thereof.

22. The method of claim 16, wherein the maleimide compound is: 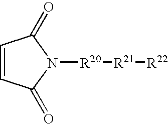
wherein R²⁰ is aryl;
R²¹ is —(OCH₂CH₂)ₙ— wherein n is 3 to 15;
R²² is a group O—R²³ wherein R²³ is aryl or C₁₋₁₀ alkyl,
wherein the aryl or alkyl group is optionally substituted with 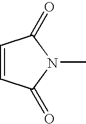
or a salt, ester or other physiologically equivalent derivative thereof.

23. The method of claim 16, wherein the maleimide compound is any one of: 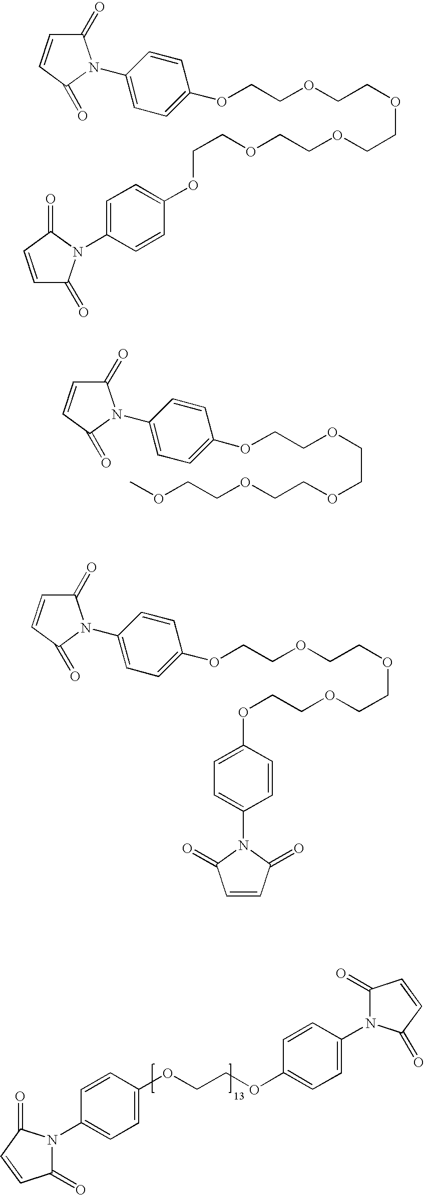
or a salt, ester or other physiologically equivalent derivative thereof.

24. A spermicidal formulation comprising a maleimide compound of formula (I): 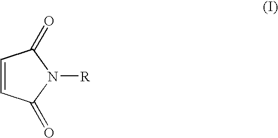
wherein R is any group;
provided in a spermicidally effective amount, or a salt, ester or other physiologically equivalent derivative thereof, with the proviso that the maleimide is not N-ethylmaleimide.

25. The spermicidal formulation of claim 24, wherein the maleimide compound is a compound of general formula (Ia): 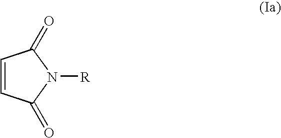
wherein:
R represents a group A, which itself is represented by (R¹)ₙ—(R²)ₚ—(R³)_{q}, wherein each of R¹, R² and R³ is independently selected from:
an acyclic aliphatic group having 1 or from 3-20 carbon atoms;
a polyalkylene glycol group having an average molecular weight in the range 100 to 1000;
a cyclic aliphatic group having from 4-20 carbon atoms;
a monocyclic or polycyclic aromatic hydrocarbon group having from 6-18 ring carbon atoms;
a non-aromatic heterocyclic group having from 3 to 8 ring atoms, or
a monocyclic or polycyclic aromatic heterocyclic group having from 5 to 20 ring atoms;
and n, p and q each represent 0 or 1 such that the sum of n+p+q=1-3;
or R represents the group A substituted by one or more groups independently selected from: 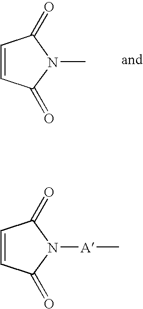
wherein A′, which may be the same as or different from A, represents a group (R¹)ₙ—(R²)ₚ—(R³)_{q}, wherein R¹, R², R³, n, p and q are as defined above;
or a salt, ester or other physiologically equivalent derivative thereof.

26. The spermicidal formulation of claim 25, wherein the maleimide compound is a compound of formula (II): 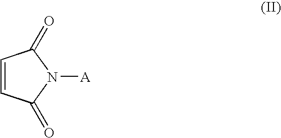
wherein A is as defined in claim 25, or a salt, ester or other physiologically equivalent derivative thereof.

27. The spermicidal formulation of claim 25, wherein the maleimide compound is a compound of formula (III): 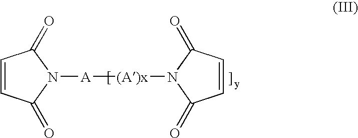
wherein A and A¹, which may be the same or different, are as defined in claim 25, x is 0 or 1 and y is 1-6, or a salt, ester or other physiologically equivalent derivative thereof.

28. The spermicidal formulation of claim 25, wherein the maleimide compound is a compound of formula (IV): 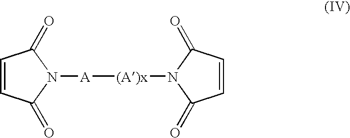
wherein A, A¹ and x are as defined in claim 25, or a salt, ester or other physiologically equivalent derivative thereof.

29. The spermicidal formulation of claim 25, wherein the maleimide compound is compound of formula V: 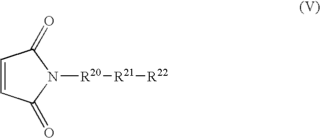
wherein R²⁰ is aryl;
R²¹ is —(OCH₂CH₂)ₙ— wherein n is 15;
R²² is a group O—R²³ wherein R²³ is aryl; or
R²¹ is —OCH₂CH₂)ₙ wherein n is 3 to 15;
R²² is a group O—R²³ wherein R²³ is C₁₋₁₀ alkyl,
wherein the aryl or alkyl group is optionally substituted with 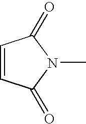
or a salt, ester or other physiologically equivalent derivative thereof.

30. The spermicidal formulation of claim 25, wherein the maleimide compound is selected from any one of: 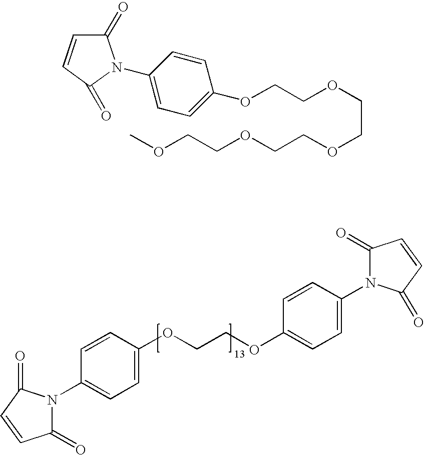
or a salt, ester or other physiologically equivalent derivative thereof.

31. The spermicidal formulation of claim 25, wherein the maleimide compound is: 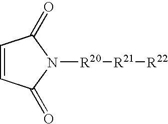
wherein R²⁰ is aryl;
R²¹ is —(OCH₂CH₂)ₙ— wherein n is 3 to 15;
R²² is a group O—R²³ wherein R²³ is aryl or C₁₋₁₀ alkyl,
wherein the aryl or alkyl group is optionally substituted with 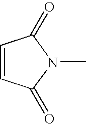
or a salt, ester or other physiologically equivalent derivative thereof.

32. The spermicidal formulation of claim 25, wherein the maleimide compound is any one of: 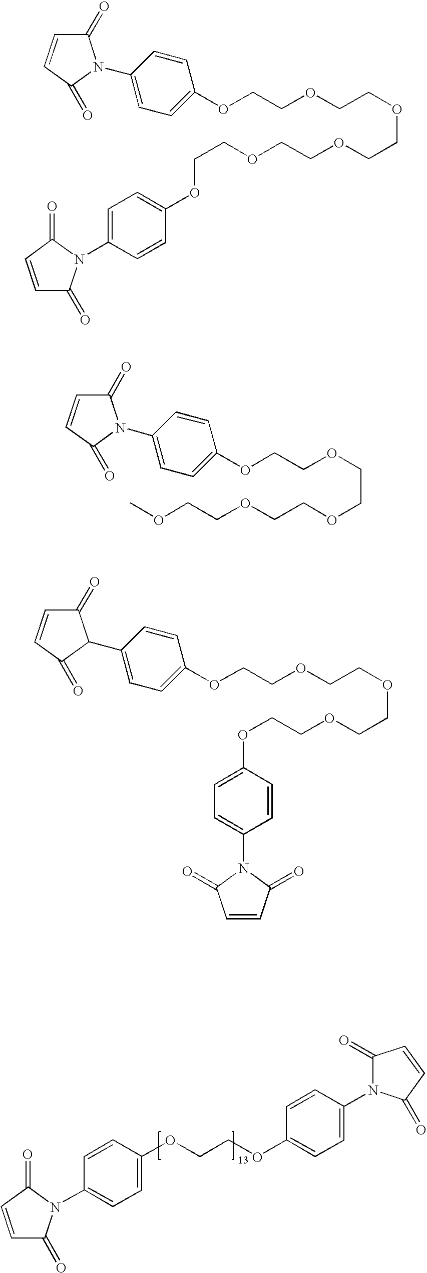
or a salt, ester or other physiologically equivalent derivative thereof.

33. A compound of formula V: 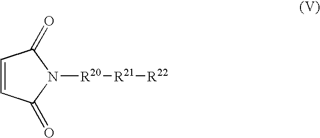
wherein R²⁰ is aryl;
R²¹ is —(OCH₂CH₂)ₙ— wherein n is 15;
R²² is a group O—R²³ wherein R²³ is aryl; or
R²¹ is —(OCH₂CH₂)ₙ wherein n is 3 to 15;
R²² is a group O—R²³ wherein R²³ is C₁₋₁₀ alkyl,
wherein the aryl or alkyl group is optionally substituted with 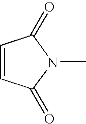
or a salt, an ester, an amide or a prodrug thereof.

34. The compound of claim 33 selected from any one of: 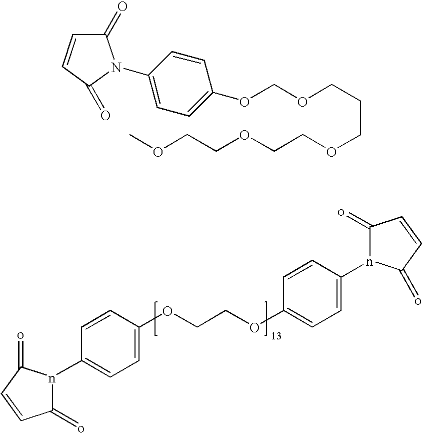
or a salt, an ester, an amide or a prodrug thereof.

35. The compound of claim 33 selected from any one of: 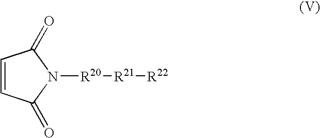
wherein R²⁰ is aryl;
R²¹ is —(OCH₂CH₂)ₙ— wherein n is 3 to 15;
R²² is a group O—R²³ wherein R²³ is aryl or C₁₋₁₀ alkyl,
wherein the aryl or alkyl group is optionally substituted with 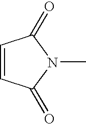
or a salt, an ester, an amide or a prodrug thereof.

36. The compound of claim 33 selected from any one of: 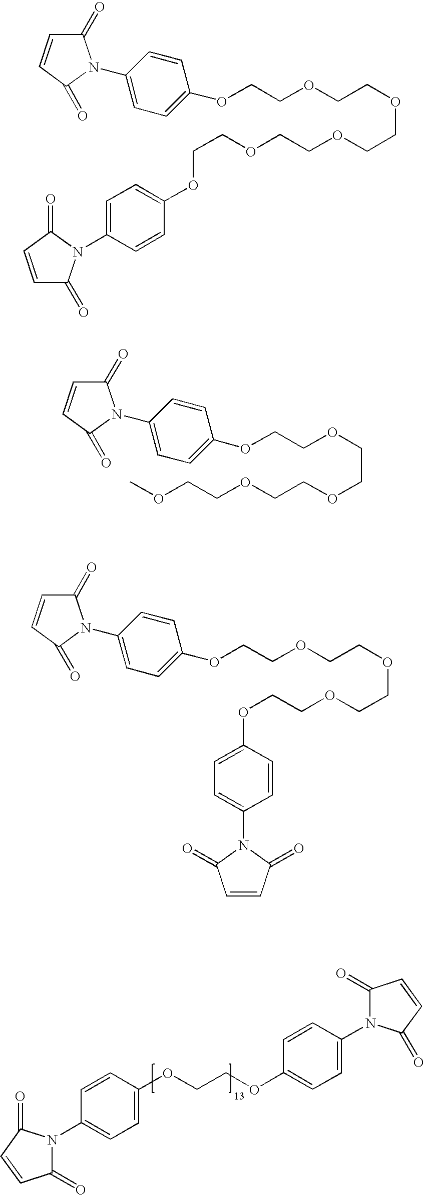
or a salt, an ester, an amide or a prodrug thereof.
